(19) United States
(12) Patent Application Publication
(10) Pub. No.: US 2019/0380951 A1
(43) Pub. Date: Dec. 19, 2019

(54) COMPOSITION, DEVICE AND METHOD FOR CONFORMATIONAL INTRA-TISSUE BETA BRACHYTHERAPY

(71) Applicant: ScintHealth GmbH, München (DE)

(72) Inventors: Maria DESANTIS, Rome (IT); Cesidio CIPRIANI, Rome (IT)

(21) Appl. No.: 16/480,266

(22) PCT Filed: Dec. 22, 2017
(86) PCT No.: PCT/IT2017/000292

§ 371 Date: Jul. 23, 2019

(30) Foreign Application Priority Data
Jan. 24, 2017 (IT) 202017000007330
Jan. 24, 2017 (IT) 202017000007344

(51) Int. Cl. A61K 9/00; A61N 5/10; A61K 47/38

(52) U.S. Cl. CPC A61K 9/0024; A61K 51/1217; A61K 47/38; A61N 5/1001

(57) ABSTRACT

It is claimed the invention of a composition and a device to be used in medical therapy as a containment matrix in conformational intra-tissue beta brachytherapy. The composition is made so as to form a gel which can be injected intra-tissue without toxicity in the organism, holding in suspension during injection the particulate of a beta-emitting brachytherapy composition, and forming after injection a solid deposit that immobilizes the radiotherapeutic composition in the injection bolus, to prevent migration of the radioactive product into the surrounding tissues. The composition is injected with an apparatus dedicated to percutaneous intra-tissue injection comprising a 7-degree robotic apparatus, an automatic injection device under pressure, a needles system to provide the therapeutic composition application in the tissue to be treated with minimal trauma. An advanced software system is also included that allows interfacing between diagnostic imaging data, robotic arm movement scheduling, and composition dose distribution, in order to optimize the distribution of the radiotherapeutic doses of the composition into the tissue to be treated, according to a targeted individual therapeutic strategy. Additionally a method for using the invention for the application in human or animal medicine is claimed.

### TECHNICAL FIELD AND DEFINITIONS

Today there are many pharmacological and radiotherapeutic protocols in oncology, that offer a good therapeutic response in many clinical cases. However, often these approaches cannot provide highly localized treatments, while side effects on the patient are not negligible. For example, the use of modern radiotherapy has reduced the amount of radiation given to the healthy tissue, but forces patients to undergo frequent treatment sessions and often does not always reach a sufficiently high target/non-target ratio. Chemotherapy, on the other hand, generally has a significant impact on the body and quality of life, and does not always ensure a radical and definitive healing as—like in the case of radiotherapy—its destructive action on the tumor is limited by its toxicity to non-tumor tissue. In order to overcome this, a trend has developed over the last years focused on targeted personalized minimally-invasive treatments that, with the help of highly reliable machines and tools, can narrow the damage to cancerous tissues at the same time minimizing (a) the impact of treatment on the patient, (b) the adverse effects on healthy tissues and (c) the waste of resources (like drugs and radioactive materials). For example, the use of loco-regional percutaneous treatment techniques is increasing currently in the treatment of both unifocal and multifocal formations of hepatocellular carcinoma or liver metastasis. Such techniques offer good results in terms of control of disease and survival, and can be used even in patients with important collateral diseases and/or in elderly people. One of the most experimented technique for which on a large number of patients results are available is percutaneous injection of ethanol. This technique is performed under local anesthesia, using thin needles under X-ray guidance or ultrasound for administration of high concentration alcohol (mainly ethanol) in the tissue to be destroyed. The alcohol results in a necrosis of the tissue that it comes into contact. This technique can be used for nodules of up to 5 cm in diameter, independent if primary, metastatic, or relapsed. The therapeutic effect with this technique is however limited to the region in which the alcohol comes into contact with tissue, which cannot be controlled easily nor predicted. It would then be desirable to extend this therapeutic action beyond the injection bolus, in order to destroy any contiguous neoplastic tissues. Another technique used for localized personalized therapy is brachytherapy, i.e. the local use of radioactive sources in the form of needles or seeds, mainly gamma or X emitters, with the intent to impart a lethal dose to the tumor with a minimal dose to the surrounding healthy tissues. This technique is sometimes highly invasive, as often a large number of needles of large diameter have to be implanted into the patient. Apart from this, in a number of cases, like tumor of inner organs, this protocol cannot be applied due to anatomical hindrance. Yet another approach of localized therapy is radioembolization. In this technique a beta-emitting isotope is injected into a branch of the portal vein in the form of microspheres. This protocol is applied to hepatic carcinoma, and, in some cases to liver metastases; unfortunately the distribution of the radioisotope is far from conformational (this understood as matching the exact shape of the tumor). As a result, a significant part of the healthy liver tissue is irradiated, as the microspheres are injected into a blood vessel, and the distribution is dominated by the blood distribution flux.

PROBLEM DEFINITION—Following this line of thought, the selective deposition of a lethal dose in tumor tissue by the use of a radioactive beta-emitter in the form of micro-particles or nanoparticles would enable the irradiation of in principle any tumor mass in a uniform and selective way minimizing at the same time the dose of surround healthy tissue. Particularly interesting for the use of this technique are, among others, the isotopes ⁹⁰Y, ¹⁶⁶Ho, ¹⁷⁷Lu, ³²P, ¹⁸⁶Re, ¹⁸⁸Re and ¹⁴⁴Ce. Most of these beta-emitters are produced in nuclear reactors by neutron irradiation of non-radioactive natural or isotopically enriched elements. In some cases the beta-emitting isotope is obtained from a suitable “isotope generators”. Examples of such systems are the one that makes use of from ⁹⁰Sr, a by-product of nuclear fission that decays to the formation of 90Y, or the systems that supply ¹⁸⁸Re, obtained by the decay of the isotope ¹⁸⁸W. Such beta-emitting isotopes can be transformed into micro-particles or nano-particles according to known general methods, either from solid particles produced separately, for example in a suitable ion exchange resin, or using polymers or biopolymers with very low toxicity, in which the radioisotope can be immobilized by chelation, or in the form of insoluble inorganic particles, or embedded in a polymer matrix, or encapsulated in structures like liposomes. With the possibility of carrying out an injection into the tumor using an injection device it is conceivable a precise administration of a beta-emitting isotope exclusively to the complete extension of the tumor (conformational administration), in order to save the healthy tissue as much as possible, while administering a dose of lethal radioactivity throughout the neoplastic mass. The technique, though highly promising as concept, entails, however, the risk that the radioactive product will result in the diffusion of the radioactivity in other organs. It would therefore be highly desirable to have a mean to avoid the diffusion of the radioactive micro-particles or nano-particles outside from the point of deposition, in order to avoid damage to surrounding healthy tissues, and to reach the lethal effect only in a limited radius around the bolus of injection. While a satisfactory target/non-target dose in radioimmunotherapy is considered satisfactory for values greater than 10, by the use of an intratissue brachytherapy with beta-emitting isotopes with absence of diffusion from the injection site, the dose ratio could reach a value of 100 and above. The possibility of a successfully use of beta-emitting isotopes in the form of micro-particles or nano-particles is therefore dependent on the possibility of immobilizing such radioactive vectors, less invasively as possible, in the injection site. Also if a system for radioactivity deposition as above described would be available, a second important limitation still hinders the application of a successful therapeutic protocol, i.e. the geometric precision with which the injection can be performed. Many forms of diagnostic treatment and therapeutic measures include percutaneous insertion of a needle into a lesion or organ; all these treatments are usually performed using a straight line trajectory under image-guidance (e.g. ecography, fluoroscopy, real-time MRI, OCT, photo-acoustic imaging, etc.). For example, in manual prostatic brachytherapy a needle advances through a rigid template under ultrasound control; if the needle fails to reach the target, it must be retracted and reinserted. In many procedures, the precision and effectiveness of therapy is limited by the deviation that can occur when the needle is inserted, and the needle deviation from its path decreases the effectiveness of the treatment. On the market are available also special flexible needles, usually consisting of a nickel titanium alloy called NITINOL, which have the ability to facilitate precise deposition and decrease invasiveness and trauma to the patient during medical procedures. However, mistakes and uncertainties introduced during an introduction using manual procedures definitely diminish the effectiveness of planned therapy. To overcome this problem, an image-guided robotic system would be desirable, to plan, trace and manipulate the entire injection device. A robot is a multifunctional manipulator designed for the movement of objects, tools or specialized devices, controlled through variable programming in order to accomplish a variety of tasks. The desired trajectory for the tip of the needle should be provided so that it does not penetrate delicate structures such as nerves, blood vessels or bones. This can be solved by using interventional imaging (e.g. ecography, fluoroscopy, real-time MRI, OCT, photo-acoustic imaging, etc.) in combination with high-quality pre-interventional imaging where most of these structures and the tumor(s) can be segmented. By merging the pre-interventional imaging data and the interventional one, high quality topographic information is available for optimal guidance of the needles by the robotic system. More than this, a predetermined morphology of injected bolus should be performed, in order to administer a conformational lethal dose of the radioactive dose to the whole volume of the tissue to be treated.

### EXPOSURE OF THE INVENTION AND PREFERRED EMBODIMENT

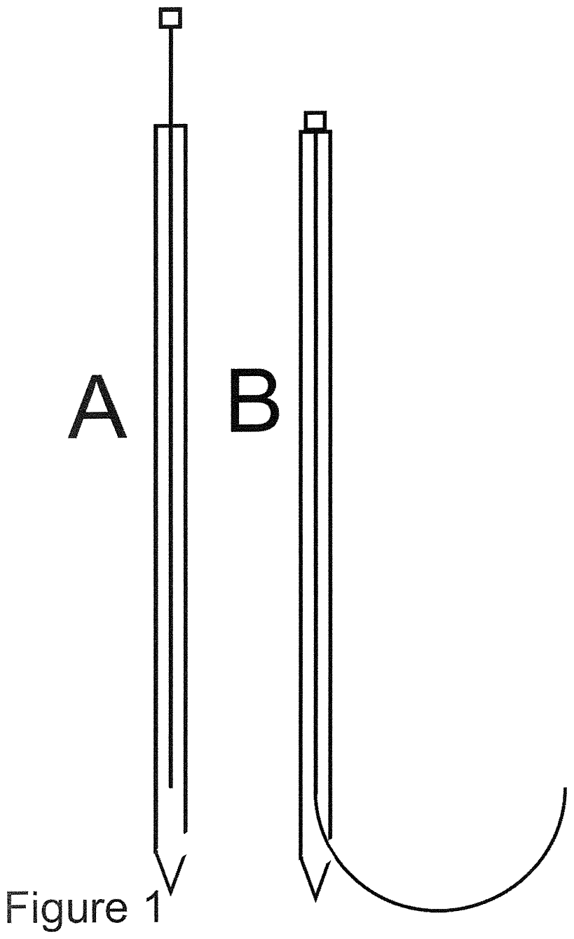

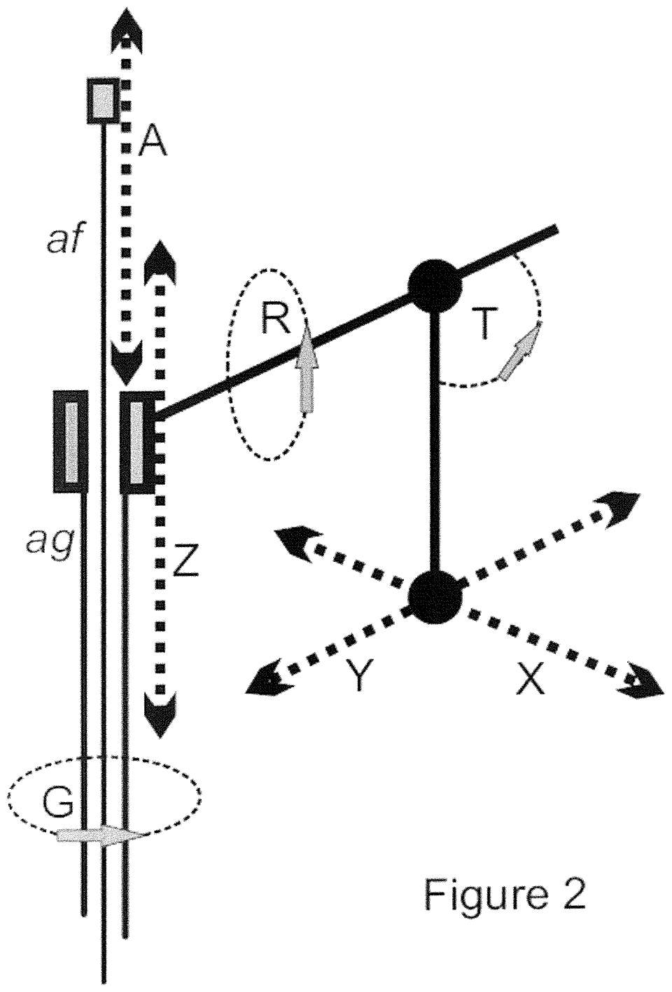

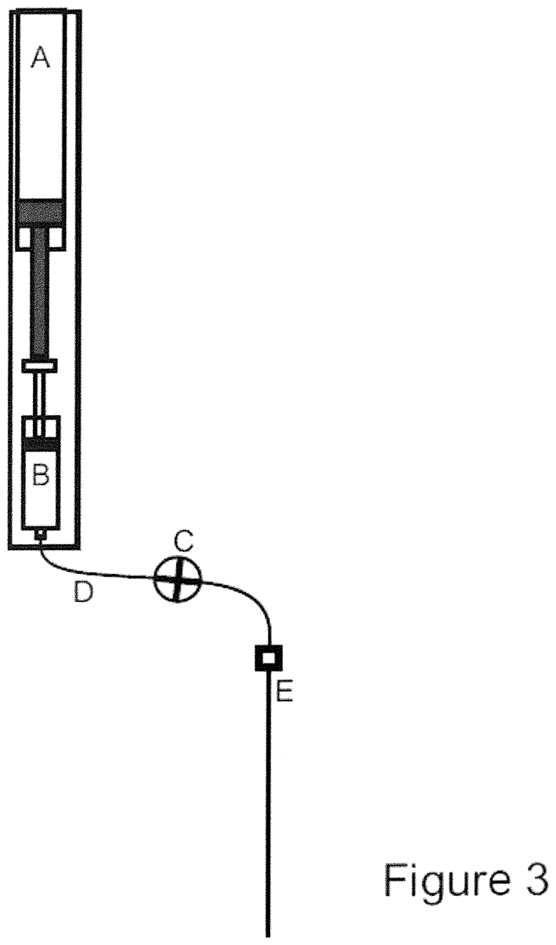

In order to realize a therapeutic protocol capable of a conformational intratissue beta brachytherapy of tumor, the present invention proposes: a combination of a composition and a device, as well as a method to apply the composition-device combination. The composition of the invention has the following characteristics: (1) can be mixed in varying proportions with radioactive nano-particles or micro-particles without any chemical or physical interaction between the composition and the said nano-particles or micro-particles (2) is capable of holding incorporated radioactive nano-particles or micro-particles, even for long periods (at least a few months) (3) has a null or negligible toxicity to human tissue and has no pharmacological effects on humans (4) it is easily administered by injection and is able to pass through an injection needle without un-mixing of composition and nano-particles or micro-particles. The composition of the present invention consists of a mixture of one or more molecules capable of forming a homogeneous lattice dispersed in ethanol having a concentration ethanol/water of 94% or greater, up to absolute ethanol (100%). The choice of ethanol as a dissolving solvent is motivated by the following peculiarities of this molecule: (1) ethanol represents a biocompatible molecule with low-toxicity for human organism (2) ethanol is currently used in clinical therapy, such as venous sclerosis, or intratissue treatment of primary hepatic tumors (3) an high concentration of ethanol produce clotting in tissue and generates cellular fibrosis, while ethanol in hydrate form is absorbed inside the cell; high concentration ethanol acts by irreversibly modifying the tertiary structure of proteins. All of these elements cause the ethanol contacting the cells to originate at the point of injection a fibrous tissue, further preventing the eventual migration of any particles suspended therein. To suspend and hold suspended particles, a polymer blend having the following characteristics is used: (1) the polymer blend is freely miscible with high concentration alcohol (2) is able to keep suspended the nano-particles or micro-particles during all the time of the injection 3) it does not show any interaction with the dispersed nano-particles or micro-particles (4) it is a composition of null or negligible toxicity, it does not exhibit any pharmacological activity and it has no appreciable interactions with the body (i.e. it is biocompatible) (5) it is able to solidify in contact with the cellular tissue, retaining the dispersed nano-particles or micro-particles inside it (6) it can be injected through an injection needle without causing any un-mixing of itself and the dispersed nano-particles or micro-particles. The polymers selected in the present invention for use in the appropriate blend are a mixture in variable ratio of (1) ethylcellulose and (2) dibenzylidene sorbitol. Both polymers have all of the abovementioned characteristics, and therefore are considered to be fully suitable for the required use. The proportions in which such polymers must be present so that the composition is optimal for the required use has been studied by systematically exploring an experimental compositional grid.—Examples of gel preparation to be used—. Example 1—100 ml of absolute ethanol are poured in a glass beaker, protected from the air. A percentage of 10% ethylcellulose and 1% dibenzylidene sorbitol is slowly added, and the solution is heated. Once the temperature of 70° C. is reached, the solution is shaken for four hours to form a homogeneous and lump-free dispersion. The resulting gel is placed in a syringe, cooled and used for subsequent tests. Example 2—100 ml of absolute ethanol are poured in a glass beaker, protected from the air. A percentage of 6% ethylcellulose and 2% dibenzyldene sorbitol is added to it, slowly adding the powder to the solution, and heating. Once the temperature of 80° C. is reached, the solution is shaken for two hours to form a homogeneous and lump-free dispersion. The resulting gel is placed in a syringe, cooled and used for subsequent tests. Example 3—100 ml of absolute ethanol are heated in a glass beaker, protected from the air. A percentage of 8% ethylcellulose is added to it, slowly adding the powder to the solution. Once the temperature of 70° C. is reached, the solution is shaken for three hours to form a homogeneous and lump-free dispersion. The resulting gel is placed in a syringe, cooled and used for subsequent tests. Example 4—100 ml of absolute ethanol are poured in a glass beaker, protected from the air. A percentage of 5% ethylcellulose and 3% dibenzyldene sorbitol is added to it, slowly adding the powder to the solution, and heating. Once the temperature of 90° C. is reached, the solution is shaken for three hours to form a homogeneous and lump-free dispersion. The resulting gel is placed in a syringe, cooled and used for subsequent tests and measurements. Example 5—100 ml of absolute ethanol are poured in a glass beaker, protected from the air. A percentage of 14% ethylcellulose is added to it, slowly adding the powder to the solution, and heating. Once the temperature of 70° C. is reached, the solution is shaken for five hours to form a homogeneous and bulk-free dispersion. The resulting gel is placed in a syringe, cooled and used for subsequent tests. The formed gel can be mixed with micro-particles or nano-particles, and can easily be injected through needles, maintaining homogeneity and stability of the dispersion. After the choice of the optimal formulation of the composition for the dispersion and injectability of the composition, the injection can be carried out both at room temperature and, in a more reproducible manner, with a thermostated syringe at a constant temperature, so as to standardize its viscosity. If necessary the invention also contemplates adding an additional component to the composition in order to enhance its visibility in imaging. For example high echoic, high density or magnetic micro-particles or nano-particles can be mixed along the radioactive micro-particles or nano-particles to solve this issue. In preparation of this invention, several experiments were performed. The composition described above was generated using the different formulations of the examples and subsequently was mixed with micro-particles or nano-particles of various kinds (radioactive inorganic precipitates such as yttrium silicate, rhenium sulfide, yttrium phosphate, iron oxide, polymer microspheres containing radioactive isotopes, ion exchange resin microspheres containing chelate radioactive isotopes). After successful mixing, it was injected for testing in several animal tissue samples (muscle, liver, pancreas, heart tissue). The radioactive beta and/or gamma radioactive isotopes used in experiments have been ^{99m}Tc, ¹⁸⁸Re, ⁹⁰Y, ³²P, ¹⁶⁶Ho; in all cases (more than 120 experiments) after injection in biological tissue no significant radioactivity (<0.005% of total radioactivity) diffusion of micro-particles or nano-particles was detected by high sensitivity counting detector, or by auto-radiographic technique, in the surrounding living tissue. In these experiments gamma-emitting isotopes were merely used to be able to easily detect any leak of the radioactive micro-particles or nano-particles from the composition into the tissue. In these experiments the goal was to quantify the leakage of the radioactivity. For each injection bolus, a sclerotic tissue sphere surrounding the solidified polymer bolus was obtained in the various tissues, which further prevents the diffusion of added particulate, even after direct washing or perfusion of the organ with a physiological solution. So the proposed invention claims as fundamental the use exclusively of ethanol in the solubilisation of the polymer, as it is the only low-toxicity alcohol compatible with an injection into the organism, and claims the use of such polymer solution exclusively as an innovative suspension and injection support in its mixture with radioactive micro-particles or nano-particles with beta-emitting isotopes. In order to solve the second critical parameter for the realization of a conformational brachytherapy, i.e. the precision, regularity and reproducibility of the injection of the mix gel/radioactive micro-particle or nano-particle composition into the living tissue, in the present invention it is claimed a device for the injection of the radioactive above described composition, constituted by a multi-parameter robotic arm, opportunely programmed to inject the radioactivity in the whole region of the tissue to be treated (active robot) or to guide an operator such that the whole region of the region of the tissue is reached (passive robot), according a predetermined strategy and geometric distribution. In the present invention a needle penetration process in two phases is proposed; a medium-stiffness, flexible needle made of NITINOL (or similar shape memory alloy) is shaped with a rectilinear section and with the terminal section, with beveled tip, of semicircular or elliptical shape. This needle is inserted inside a second straight needle, (called guide needle), with inner diameter larger than the flexible needle outer diameter; when the flexible needle is inside the guide needle, it is forced to assume a straight shape. When the flexible needle is fully inserted into the guide needle, it assumes a straight shape (FIG. 1 A), while when penetrates into the tissue leaving the needle guide it resumes its circular or elliptical proper shape (FIG. 1B) and penetrates in the tissue along a curve circular or elliptical. The guide needle is inserted by a robotic arm (either actively—i.e. the robots inserts it automatically, or passively—i.e. the robot position the needle in the right trajectory but a user inserts it manually), following a pre-determined optimized trajectory and preferably using image-guidance from interventional imaging (e.g. ecography, fluoroscopy, real-time MRI, OCT, photo-acoustic imaging, etc.), up to the position in which the tip of the guide needle is into place within the body. In the second phase of the injection, the puncture with the flexible needle can start, that comes out of the tip of the guide needle. When the flexible needle exits the needle guide it follows in its motion a fixed curve trajectory, essentially dependent only on predefined shape, and from proper characteristics of mechanical structural stiffness of the flexible needle. The flexible needle is inserted laterally to the axis of the guide needle, through the tissue and up to the periphery of the mass to be treated. It should also be specified that the needle-tissue interaction in the pre-puncture phase corresponds to a visco-elastic behavior, while in post-puncture the forward displacements are due to the combined effects of the cutting force, friction, and tissue relaxation; finally, during retraction of the needle from the tissue, friction is the only relevant force. For this reason, only once that the flexible needle has come into place up to the periphery of the tissue to be treated, the real injection of the composition is performed, during the retraction of the flexible needle. In such a way, only during the retraction of the flexible needle a regular stream of composition is ejected from the tip of the flexible needle into the tissue; as the flexible needle is retracted into the guide needle by a stepper motor, or similar device, at regular, accurately calculated and predetermined controlled speed, the deposition of the composition into the tissue is extremely regular and reproducible. Deposition must not be necessarily continuous, but can also be discrete in form of droplets—this will depend on the planned injection protocol. By fully retracting the flexible needle into the guide needle, rotating the needle guide and repeating the flexible needle penetration operation, it is possible to obtain a second circular curve with a regular deposition of the composition, on a different plane, and so on, until a complete 360 rotation degrees of guide needle is obtained, thus describing a curved rotation plane with the flexible needle. This injection phase requires by the motors of the robotic arm the coordinated control of three degrees of freedom: the distance of insertion of the guide needle, the exit length of the flexible needle, the needle guide rotation. The shape of such an injection sequence would be similar to a funnel. The same process is repeated after retracting the guide needle into the tissue for a suitable distance; in this way the process can be repeated until homogeneously filling an entire volume of tumor morphology with a series of curved planes. The flexible needle extraction measure determines the radius of rotation of the curved plane itself; in theory then iterating this process a tumor mass of any volume and shape can be filled, approximating it with a family of curved planes. Also different curvatures can be used for the flexible needle. It is important to mention that interventional image-guidance is highly recommendable during the administration, as breathing, heart-beat and patient possible movements can result in the anatomy including the tumor(s) moving, and may make it necessary to correct the injection protocol. Embodiments of this invention also include image-guidance to compensate for this. Image-guidance can be either be performed by displaying interventional image information to a user and letting him/her correct the injection protocol or also automatically letting a software correct the injection protocol. Furthermore, if the used interventional imaging modality does not allow proper visualization of the tumor(s) or of vital structures that should be avoided like blood vessels or sensitive surrounding organs, pre-interventional imaging can be used (e.g. CT, contrasted CT, MRI in any of its protocols and with/without contrast medium, PET, SPECT, 3D ultrasound, contrast-enhanced 3D ultrasound, etc) and merged with interventional imaging through image-registration to include this pre-interventional information in the injection protocol and its correction during the intervention—Examples of realization of robotic arm and injection device for the claimed composition to be used.—The robotic system and device for injection of the claimed composition can be practically realized by modification from a plurality of industrial robotic arms currently present on the market. In order to perform all necessary characterization on lab scale on simulated tissue, and on organs ex-vivo, a complete robotic apparatus and an injection device has been realized which confirms the plausibility of the invention. It is understood that the here described device is presented herein in terms of a typical realization, but it is possible for the skilled person of the art to make substitutions, omissions and non-essential changes in project, design and realization without altering the essential characteristics of the apparatus and the spirit specific of the invention. The apparatus claimed in the invention comprises (see FIG. 2) an injection arm whose position is determined by (1) a linear axis of advancement on the X axis, called X, (2) a linear axis of advancement on the Y axis, called Y, (3) an inclination axis with respect to the X axis, called T, (4) an axis of rotation of the arm on its axis, called R, (5) a linear axis advancing along the direction Z, called Z, (6) an axis of rotation of the arm on Z axis, called G, (7) a second linear axis advancing along the direction Z, called A. The degrees of freedom X, Y , T, R are used to guide the end of the guide needle to the best position for its introduction into the tissue to be treated, the Z axis is used to move the needle guide back and forth for its insertion and retraction into the tissue, the G axis is used to rotate the guide needle, and hence the flexible needle, inside the tissue, and axis A is used to advance and retract the flexible needle within the mass to be treated; an automatic injection device inject the composition into the tissue during the flexible needle retraction phase. Let us consider a patient lying on an operating table, defined as a coordinates horizontal plane. Axes X and Y allow the entire unit to be moved horizontally, with respect to the operating table. The T axis allows tilting of the arm of a predetermined angle with respect to the X axis. The R axis allows rotation of the arm of the apparatus around its axis. The Z axis allows the advance of the guide needle, located at the end of the arm, forward or backward, once that the X, Y, T and R axes have been fixed the best position for the introduction of the same needle guide. This Z axis controls the introduction and extraction of the guide needle into the tissue. The G axis allows the rotation of the guide needle once it is inserted into the tissue to be treated. The movement of this axis is automatically deactivated by an electric switch when the flexible needle is not fully inserted within the guide needle; this prevents any rotation of the needle guide when the flexible needle is inserted into the tissue, so avoiding accidental lacerations of the tissue itself. Finally, the axis A controls the movement of the flexible needle in the guide needle, back and forth, so penetrating and retracting from the tissue to be treated, when the needle guide is inserted into the tissue. Therefore, the typical operating sequence, referring to FIG. 1 and FIG. 2 is: (1) X movement, Y movement, T rotation, R rotation; the guide needle (ag in FIG. 2) is placed in the position and with the angles provided for optimum insertion into the body. In this step, the flexible needle (af in FIG. 2) is completely retracted inside the guide needle (FIG. 1A). (2) Movement Z axis forward; the needle guide penetrates into the tissue to be treated, up to the maximum calculated depth. (3) Forward A axis movement; the flexible needle exits laterally to the guide needle (FIG. 1B) for a length P and penetrates into the tissue to be treated by following a curved trajectory until it reaches the extreme periphery of the tissue to be treated. (4) Moving A axis back; the flexible needle retracts from the tissue with a curved trajectory, and at the same time the automatic injection device activates the composition injection, as long as the flexible needle is completely inside the guide needle. At this point the injection of the composition stops. (5) Movement of rotation axis G; the guide needle, and the flexible needle that is completely reinserted into the guide needle, rotates in the tissue of X-degree angle. (6) Points 3, 4 and 5 are repeated, to a complete round angle, for (360/X) times, thus generating a set of curves that lie on a curved rotation plane. The short-distance therapeutic effect of the beta radioactive composition injected along these curves generates a sort of “rotating solid” of necrotic tissue, of thickness F, where F is the distance to which the composition is therapeutically effective. The diameter of this rotation plane depends from the length P mentioned in the previous point 3, while the angle X is chosen according to the diameter to be treated, so that tissue destruction is ensured in the space between two contiguous injection points even at the extreme periphery of the same tissue (distance D on the circumference of the rotation plane). The larger the diameter to be treated the lesser is the X angle. (7) Z back movement; the flexible needle is completely retracted into the guide needle and the needle guide is retracted from the tissue to be treated of a distance F, to continue processing with a second curved rotation plane; such distance F is usually of a value similar to distance D of the previous point 6. (8) Points 3, 4, 5, 6 are repeat again, thus generating a second “rotating solid” of necrotic tissue, parallel and adjacent to the first, with a diameter that may also be different from the first plane of rotation. If the tissue to be treated has not a circular geometry, the flexible needle can penetrate for each single injection of a different length, thus generating a curved plan with an elliptical section conformated to the morphology of the tumor that is being treated. (9) Point 7 and again points 3, 4, 5, 6 are repeated until all the tumor is completely filled by the “rotating solids” of necrotic tissue; at this point, the flexible needle returns into the guide needle, and the same needle guide is retracted from the body, leaving the patient. The treatment is finished. The automatic device for the injection of the composition is composed of a syringe containing the composition to be injected (FIG. 3), closed by a low-volume rotary valve (D in FIG. 3) controlled by a stepper motor. The syringe is subjected to constant and controlled pressure, for example by pushing the piston of the syringe by a compressed gas piston with constant pressure (A in FIG. 3), or similar apparatus. When the rotary valve is open, a certain amount of composition is ejected from the syringe under pressure through a connected small flexible tube of TEFLON or PEEK (C in FIG. 3), connected to the flexible injection needle (E in FIG. 3), and is injected into the tissue; the syringe is suitably thermostated to ensure that the composition has a constant viscosity. Once the injection apparatus with connection tube and injection needle is mounted, the amount of composition ejected from the syringe and injected from the needle is only a function of the opening time of the valve, and this amount can be easily controlled and rendered constant. During injection and until treatment is completed, the needle position and the injected radioactive composition are constantly displayed with an imaging system by a mini gamma camera (to visualize the gamma-emissions or the Bremsstrahlung generated by the beta-emitting isotope), and/or anatomical imaging devices like fluroscopy device, or a high resolution ultrasonic apparatus, measurement of absolute movement coordinates with an optical scanner in order to know the relative position and orientation of the imaging devices with respect to the robotic injection system, so as to allow for a constant monitoring of the treatment by the operators. For the sake of plausibility evaluation, the prototypic robotic apparatus built has been used in a large number of injection experiments on several different animal tissue samples (muscle, liver, pancreas, heart tissue). The morphological distribution of the composition into the tissue has been found to be closely following the predetermined curved trajectory of the needle. In some cases, instead of using the flexible needle, an ordinary straight needle has been used for injection, using only the X, Y and Z axis of the robotic arm for positioning of the needle, and employing the same injection apparatus. In this case a multi-hole pattern of the radioactive composition injections have been obtained in the biological tissue. Although there is the disadvantage of a multi-hole injection, this variation of the technique allowed the use of needles of much smaller diameters (from 20 G up to 30 G), with lesser traumatism on the body, with controlled morphology and without any diffusion of radioactivity outside from injection sites.

ACTUAL STATE OF ART—The use of ethylcellulose, which constitutes the fundamental polymer in the composition of the present invention, has various applications in technology and medicine; for example it is currently used for the coating of pills, or as a food additive, or in the creation of oily dispersions. The ethylcellulose and ethanol gel therefore constitutes only a dispersion matrix in the proposed invention, and only its properties are exploited to rapidly solidify within a living tissue, immobilizing it, the therapeutic medium within the solid formed in the tissue. Patents U.S. Pat. No. 8,101,032 (B1) and KR20150065301 (A) describe a preparation wherein methylcellulose is used as a gelling agent of alcohol as a fuel for chemical rockets; no mentioned is made in the patent to the properties of an alcoholic gel for medical use, and no claim has been advanced in that field—also these patents do not mention the embedding of radioactive micro-particles or nano-particles in the said gelling agent. Patent ES2049660 (A1) describes a gel for use in medicine but with a formulation totally different from that claimed herein and with the intent of constructing a product with vaso-constricting effect on the veins. In the formulation there is no indication of the use of ethylcellulose as a dispersion medium in a biological tissue nor any hint of using radioactive micro-particles or nano-particles dispersed in the gel. The patent JPS5869248 (A) describes a gel for use in medicine, but with a formulation totally different from that claimed herein, and with the intent to construct a product for external skin application. A carboxyvinyl polymer and a soluble nylon are used in the composition, and no mention is made of the use of ethylcellulose as a polymer nor any hint of using radioactive micro-particles or nano-particles dispersed in the gel. Patent WO2016010741 (A1) describes the use of an aqueous dispersion of ethylcellulose for forming film coatings; no mention is made in the patent to the properties of the alcoholic gel in medicine, and no claim has advanced in that field—also these patents do not mention the embedding of radioactive micro-particles or nano-particles in the said gelling agent. The patent WO2014193667-(A1) describe a process for preparing an oleogel from ethylcellulose; in the described and claimed composition no mention is made of the use of an ethanol solution, and of any application as injective media in therapeutic application—also these patents do not mention the embedding of radioactive micro-particles or nano-particles in the said gelling agent. In patent DE3814910 (A1) it is described a process for the preparation of lipoid ethylcellulose gels and pharmaceutical, cosmetic and industrial use. In the formulation of the product therein claimed only fatty alcohols, castor oil, paraffins or fatty esters (waxes) can be added to the solution. In the said patent only the use of alcohol with long chain is explicitly and precisely mentioned and claimed, with chain from C8 to C18, (fatty alcohols, mainly used in cosmetic field), and no mention is made of a solution of ethylcellulose in absolute ethanol, no mention of the use of the obtained composition in dispersing radioactive micro-particles or nano-particles for therapeutic uses, and no mention of the use of the obtained composition for injection of the mix polymer/particulate in a biological tissue. Percutaneous infusion procedures may be subdivided in two categories: (1) inserting a rigid needle through the skin and soft subcutaneous tissues in a precise position inside the body; sometimes such rigid needle may be a guide needle, inside which there may be a second flexible needle for the actual injection (2) procedures where a guide catheter wire is inserted into a blood vessel and is used as a channel to place a tool at the end of the same catheter into a tissue inside the body. The catheters are generally larger than the needles, are usually inserted into a fluid and open space inside the body, and their distal tip can be manipulated with a minimum resistance. Percutaneous needles are typically used to make a soft tissue biopsy or ablation. Sometimes the needles are designed to be inserted into a tissue and be guided into the tissue itself. This solution is definitely more complex than the use of the catheter, but causes much less traumatism in the body. Numerous patents have been claimed on methods for driving a flexible needle within a living tissue. The simplest method is to bend a flexible needle so that it follows a curved trajectory when inserted into the tissue. A leverage of the asymmetric needle tip can also be used to produce a lateral deviation in the needle. U.S. Pat. No. 5,938,635 and US 2004/0133168 propose for this purpose the use of concentric needles; even though it has demonstrated the ability to actually guide the needle into standard tissue, this approach actually involves knowledge of tissue properties, especially with the use of relatively thin needles. In this regard, US patents 2007/0167868 and U.S. Pat. No. 5,318,528 make use of appropriately shaped cutting surfaces to guide the orientation of a needle into the body. Several adjustable needles on the market today are COOK Pakter Curved Needle Set, COOK Osteo-Site Bone Access, PneumRx Seeker Biopsy Needle. The first two patents use pre-curved needles inside a guide cannula, while the third patent carries out the needle curvature by means of a tilting knob acting on four sheets of steel which curl the needle through a mechanism operated by hand by the doctor. All of these devices and the aforementioned patents are essentially based on the operator's manual ability and lack of accurate controllability, particularly when the needle is already partially inserted into the tissue. Furthermore, there is no locking mechanism for them to hold a particular curvature, nor any automatic system for advancing the same needle in the tissue. Finally, there is not a safety mechanism that prevents the cannula rotation when the needle is inserted, thus leading to the risk of accidental tissue tearing during the guide needle movement. U.S. Pat. No. 6,592,559 B1 claims a device consisting of a cannula including a second superelastic needle such as NITINOL. The needle is machined to produce a preformed curve that can be straightened by passing through the coaxial outer cannula, when introduced into a patient's body. Leaving from the outer cannula, the inner needle essentially resets the preformed configuration for the introduction or extraction of materials in lateral areas to the guide needle entry path. U.S. Pat. No. 6,425,887 claims a device consisting of an infusion cannula which includes a plurality of super-elastic needles like NITINOL. The needles are machined to produce a preformed curve that can be straightened by passing through the coaxial outer cannula, while introducing into a patient's body. Outside the outer cannula, the internal needles return substantially to the preformed configuration for the introduction or extraction of materials in lateral areas to the needle group path. U.S. Pat. No. 6,572,593 discloses a device consisting of a deformable catheter placed within a rigid cannula. The device catheter is bent at the distal end and can be rotated axially within the cannula lumen so as to provide a simple maneuverability for precise positioning of the catheter. The catheter is made of a material that maintains its curved shape when extracted from rigid cannula. In the last three patents the insertion and positioning control is purely manual and there is no mention of a device control with an automatic or semiautomatic apparatus or a coupling with the coordinates of the medical images so as to precisely address a volume of tissue to be treated. Patent WO 2007/141784 A2 claims a robotic system for guiding a flexible needle during insertion into a soft tissue using images to determine the needle position. The control system calculates a needle point trajectory to the desired target, avoiding potentially dangerous obstacles along the path. Using an inverse kinematic algorithm, the required maneuvers are calculated in such a way that the needle follows a trajectory in the tissue to be treated. While the patent introduces the concept of robotic system, no claim is made on the ability to handle predefined volumes by using a strategy of filling a given volume with a treatment plan. Furthermore, there is no claim for any system capable of effectively interacting between robotic and medical imaging systems in order to minimize patient trauma and treatment times and optimize a therapeutic strategy on tissue volume to be treated, and no claim on the complete therapeutic filling of the volume to be treated by an integrated robotic arm and an injection automatic system. U.S. Pat. No. 5,792,110 claims a system and a method to place therapeutic agents on specific tissues in a subject to be treated. The system allows precise positioning of a selected amount of therapeutic agent in a three-dimensional matrix of a predetermined site in a subject to be treated with minimal trauma. The system comprises a guide cannula to penetrate a selected tissue at a predetermined depth, and a second cannula for delivering the therapeutic agent to the subject. The guide cannula has an axial hole with an open proximal end and an opening at the distal end. The delivery cannula has a flexible portion at the distal end passing through the hole of the first cannula, and an outside diameter which is less than the inner diameter of the guide cannula. In this patent, the delivery cannula is flexible but not preformed; it is displaced by a deviation at the distal end of the outer cannula, so it remains straight as it is inserted into the tissue. Thus the maximum range of action is strictly limited, and its geometers are inaccurate. Also in this patent the cannula is to be inserted manually and no mention is made of a device control such as a tele-robot or a coupling with the coordinates of medical images so as to precisely address a volume of tissue to be treated. US 2006/0229641 A1 discloses a method and device for guiding and inserting a tool into an object, such as a biological tissue. In an embodiment, a guide device is provided that can be controlled remotely to adjust the insertion of a tool along a path and to move the tool into the tissue to the desired depth of penetration. The instrument can be, for example, a biopsy device, a device for brachytherapy, or a surgical device. The device can be configured for use with an imaging device, such as computerized tomography (CT), to allow the instrument to be positioned accurately. While the patent introduces the concept of a servo-controlled system, no claim is made on the ability to handle predefined volumes by means of a homogeneous filling strategy of a given volume of variable morphology. Furthermore, no system capable of interacting between robotic systems and data from medical imaging procedures is claimed to minimize patient trauma and treatment times, and optimize a therapeutic strategy on tissue volume to be treated, and no mention is made of an automatic injection system. Finally, more importantly, the device works for rectilinear trajectories and is unable to reposition the distal tip of the medical instrument after it has been inserted into the tissue to be treated

1. Composition for conformational intra-tissue beta brachytherapy essentially composed of ethanol, ethylcellulose, dibenzylidene sorbitol, and at least one beta-emitting isotope in the form of micro-particles or nanoparticles, in organic or inorganic form.

2. Composition for conformational intra-tissue beta brachytherapy according to claim 1, in which the ethylcellulose is comprised between 4% and 16% by weight of the total, preferably between 6% and 14.

3. Composition for conformational intra-tissue beta brachytherapy according to claim 2, in which the ethanol is comprised between 80% and 97% by weight of the total, preferably between 82% and 94%.

4. Composition for conformational intra-tissue beta brachytherapy according to claim 3, in which dibenzylidene sorbitol is present in variable amounts, not exceeding 4% by weight of the total.

5. Composition for conformational intra-tissue beta brachytherapy according to claim 4, in which the isotopes in form of micro or nano particles of the composition are preferably chosen in the group of ⁹⁰Y, ¹⁶⁶Ho, ¹⁷⁷Lu, ³²P, ¹⁸⁶Re, ¹⁸⁸Re, ¹⁴⁴Ce.

6. Composition for conformational intra-tissue beta brachytherapy according to claim 5, treated in order to be sterile and pyrogen-free, into a ready-to-use syringe for its use in therapeutic treatment in human and/or animal therapy, by percutaneous and/or intra-operatory intra-tissue injection.

7. Composition for conformational intra-tissue beta brachytherapy according to claim 6 wherein the beta-emitting isotope is one or a mix of the following ⁹⁰Y, ¹⁶⁶Ho, ¹⁷⁷Lu, ³²P, ¹⁸⁶Re, ¹⁸⁸Re and ¹⁴⁴Ce.

8. Composition for conformational intra-tissue beta brachytherapy according to claim 7 where the beta-emitting isotope is in form of micro-particles or nanoparticles preferably rhenium-sulphide, rhenium-oxide, rhenium-sulphur colloid, metallic rhenium, yttrium -silicate, yttrium-phosphate, yttrium-oxide, yttrium-fluoride, yttrium-oxalate, yttrium-hydroxide or any combination of them.

9. Apparatus for conformational intra-tissue beta brachytherapy for injection of the therapeutic composition, according to claim 1, dedicated to a conformational brachytherapy intra-tissue treatment, in which a straight needle is connected to the syringe containing the radioactive composition, and is used for percutaneous injections into the tissue to be treated.

10. Apparatus for conformational intra-tissue beta brachytherapy according to claim 9, in which the syringe is connected, by a flexible tubing, to a flexible needle having a first section with a rectilinear elongated shaft (proximal section) and a second section (distal section) of semicircular or elliptical shape, ending with a beveled tip.

11. Apparatus for conformational intra-tissue beta brachytherapy according to claim 10, in which the flexible needle can be made up with a superelastic shape memory alloy, preferably NITINOL.

12. Apparatus for conformational intra-tissue beta brachytherapy according to claim 10, where the syringe containing the composition to be injected can be connected through a low dead-volume rotary-valve, normally closed, to the flexible tubing connected to the flexible needle.

13. Apparatus for conformational intra-tissue beta brachytherapy according to claim 12, in which the syringe containing the composition to be injected can be kept under a constant controlled pressure, for example by pushing the piston of the said syringe by the piston of a compressed gas piston or an analog device, and the same syringe can be thermostated at a predetermined temperature to keep constant the viscosity of the contained composition.

14. Apparatus for conformational intra-tissue beta brachytherapy according to claim 13 in which the opening of the rotary-valve can allow the release of the composition from the pressurized syringe to the injection flexible needle in controlled amounts.

15. Apparatus for conformational intra-tissue beta brachytherapy according to claim 14 in which the flexible needle can be inserted and enclosed within a straight guide needle having an inner diameter larger than the outer diameter of the flexible needle, and in this position the same flexible needle is forced to assume a straight configuration.

16. Apparatus for conformational intra-tissue beta brachytherapy according to claim 15, in which the guide needle enclosing the flexible needle can be used for the percutaneous insertion, following a straight path, in the body, to reach the tissue to be treated.

17. Apparatus for conformational intra-tissue beta brachytherapy according to claim 15, in which, when the tip of the guide needle is in position within the tissue to be treated, the flexible needle can be pushed outside from the tip of the guide needle and can deflect laterally to the axis of the same guide needle, so penetrating into the tissue to be treated.

18. Apparatus for conformational intra-tissue beta brachytherapy according to claim 17 in which the flexible needle penetrating into the tissue follows the trajectory of the proper shape of its distal section, with a shape semicircular or elliptical.

19. Apparatus for conformational intra-tissue beta brachytherapy according to claim 18 in which the flexible needle is pushed in order to penetrate into the tissue up to the periphery of the mass to be treated, and is then pulled back, in order to be retracted into the guide needle, by following the same trajectory of its penetration, but in the opposite direction.

20. Apparatus for conformational intra-tissue beta brachytherapy according to claim 19 in which, in the moment in which the flexible needle is starting its retraction into the guide needle, the rotary valve is opened, allowing the composition contained in the syringe under pressure to flow through the flexible needle into the tissue to be treated.

21. Apparatus for conformational intra-tissue beta brachytherapy according to claim 20 in which, after completion of deposition of the composition along the path of the flexible needle and total and complete retraction of the flexible needle into the guide needle, the guide needle can be rotated into the tissue to be treated of a predetermined angle.

22. Apparatus for conformational intra-tissue beta brachytherapy according to claim 21 in which a complete cycle of penetration of flexible needle, retraction of flexible needle and therapeutic composition deposition, rotation of guide needle can be repeated a number of times, performing a complete 360° degrees guide needle rotation in order to complete the conformational deposition of the radioactive composition in the first plane of tissue to be treated.

23. Apparatus for conformational intra-tissue beta brachytherapy according to claim 22 in which the needle guide is retracted from the tissue of a predetermined length, and the complete cycle can be repeated, up to a complete conformational deposition of radioactive composition into the whole volume of tissue to be treated.

24. Apparatus for conformational intra-tissue beta brachytherapy according to claim 9, in which the complex of all the injection apparatus is fixed to the hand of a robotic arm having at least a total of 7 degrees of freedom (among linear axes and rotation axes) so to assume the best geometric orientation of the guide needle for its insertion into the tissue to be treated.

25. Apparatus for conformational intra-tissue beta brachytherapy according to claim 22, where a linear motion of the robotic arm is used for insertion and retraction of the guide needle, and of the enclosed flexible needle, into the tissue and from the tissue to be treated.

26. Apparatus for conformational intra-tissue beta brachytherapy according to claim 25, in which a rotation motion of the robotic arm along the same axis of the guide needle is used for rotation of a predetermined angle of the guide needle, and of the enclosed flexible needle, within the tissue to be treated, exclusively when the flexible needle is completely retracted and enclosed into the guide needle.

27. Apparatus for conformational intra-tissue beta brachytherapy according to claim 26, in which a second linear motion of the robotic arm is used (a) for the penetration of the flexible needle, out from the tip of the guide needle, into the tissue to be treated, and (b) for the retraction of the flexible needle from the tissue into the needle guide, during the phase of injection of the composition in the tissue itself.

28. Apparatus for conformational intra-tissue beta brachytherapy according to claim 27, in which the robotic arm, the automatic injection apparatus and the needle insertion apparatus are constituted in a single coordinated device, integral with to the operating table on which the patient is placed.

29. Apparatus for conformational intra-tissue beta brachytherapy according to claim 27, in which the robotic arm, the automatic injection apparatus and the needle insertion apparatus are controlled by an electronic system constituted in a single block, programmable by a series of coordinates derived from a therapeutic conformational strategy, morphologically calculated for each lesion to be treated.

30. System for conformational intra-tissue beta brachytherapy according to claim 29, where the coordinates and parameters of the program controlling the motions of the automatic injection apparatus and of the needle insertion robot are derived by the use of a software interface which, by comparative analysis of the images from different medical diagnostics (ultrasound, CT, PET, RM, scintigraphy), is able to calculate the best motion strategy to minimize guide needle injection damage, optimize homogeneity and reproducibility of deposition of radioactive therapeutic doses in the tissue to be treated, minimizing treatment times.

31. System for conformational intra-tissue beta brachytherapy according to claim 30, where the needles positions and the injected radioactive composition are constantly displayed to the medical operators in a imaging systems that combines interventional image-guidance preferably using a gamma camera, fluoroscopy, ultrasound, real-time MRI, OCT, photo-acoustic imaging or any combination of them, so that medical operators can constantly monitor and validate the treatment during the injection stage, and until the treatment completion.

32. System for conformational intra-tissue beta brachytherapy according to claim 31, where pre-interventional imaging information preferably CT, contrasted CT, MRI in any of its protocols and with/without contrast medium, PET, SPECT, 3D ultrasound, contrast-enhanced 3D ultrasound or any combination of these is registered to the interventional image-guidance.

33. Method for conformational intra-tissue beta brachytherapy consisting in: (a) defining a tumor target region, (b) defining an injection protocol such that the complete tumor area is reached by a needle, (c) injecting a needle with the apparatus according to claim 9, (d) depositing the composition for conformational intra-tissue beta brachytherapy, for the application in human or animal medicine.

34. System for conformational intra-tissue beta brachytherapy according to claim 30, wherein the system is configured to implement a method for conformational intra-tissue beta brachytherapy consisting in: (a) defining a tumor target region, (b) defining an injection protocol such that the complete tumor area is reached by a needle, (c) injecting a needle with the apparatus for conformational intra-tissue beta brachytherapy, (d) depositing the composition for conformational intra-tissue beta brachytherapy, for the application in human or animal medicine.
